(19) United States
(12) Patent Application Publication
(10) Pub. No.: US 2024/0003917 A1
(43) Pub. Date: Jan. 4, 2024

(54) Non Invasive Device for Early Stage Alzheimer's and Neurodegenerative Disease Detection

(71) Applicant: Richard Postrel, Miami Beach, FL (US)

(72) Inventor: Richard Postrel, Miami Beach, FL (US)

(21) Appl. No.: 18/244,906

(22) Filed: Sep. 11, 2023

Related U.S. Application Data
(63) Continuation of application No. PCT/US22/19841, filed on Mar. 10, 2022.
Application No. PCT/US22/19841 is a continuation-in-part of application No. 17/692,073, filed on Mar. 10, 2022, now abandoned.
(60) Provisional application No. 63/160,789, filed on Mar. 13, 2021.
(60) Provisional application No. 63/158,903, filed on Mar. 10, 2021.
(60) Provisional application No. 63/158,903, filed on Mar. 10, 2021.
(60) Provisional application No. 63/160,789, filed on Mar. 13, 2021.

(51) Int. Cl. G01N 33/68; A61B 5/00; A61B 10/00

(52) U.S. Cl. CPC G01N 33/6896; A61B 5/4088; A61B 10/00

(57) ABSTRACT

A disease, by definition, is an undesired or abnormal state. Diseases are characterized by symptoms that may change during disease progression. But each symptom is underpinned by at least one factor that alters metabolism within cells of the body. Without at least one change in cell activity (metabolism) there would be no symptoms. These metabolic alterations involve modified rates or occurrence of alternate biochemical reactions. Disease-associated patterns of reactions (metabolisms) produce a characterizing or signature pattern of reaction products and byproducts. These metabolites, especially volatile organic metabolites (VOCs), may be assayed using a device of this invention to produce results that indicate a subject's disease or class of diseases and to differentiate between diseases.

This invention specifically relates to Alzheimer's disease and other neurodegenerative diseases. The present invention additionally features devices enabling non-invasive detection of autoimmune diseases using Volatile Organic Compounds (VOCs) from inner ear gases and earwax. Diseases including, but not limited to: Multiple Sclerosis, Alzheimer's and Parkinson's Disease can be detected through characteristic VOC signatures. One or more devices of this invention are instrumental in analyzing disease specific collections of volatile organic compounds (VOCs) found in the otic canal either in gaseous form or captured in a solid form from the secreted protective surface of the canal. The invention discloses use of the invented devices to produce signatures and/or profiles of VOC contents that are illustrative of the presence or absence of a particular disease.

A disease, by definition, is an undesired or abnormal state. Diseases are characterized by symptoms that may change during disease progression. But each symptom is underpinned by at least one factor that alters metabolism within cells of the body. Without at least one change in cell activity (metabolism) there would be no symptoms. These metabolic alterations involve modified rates or occurrence of alternate biochemical reactions. Disease-associated patterns of reactions (metabolisms) produce a characterizing or signature pattern of reaction products and byproducts. These metabolites, especially volatile organic metabolites (VOCs), are assayed using a device of this invention to produce results that indicate a subject's disease or class of diseases and to differentiate between diseases.

The underlying concept is confirmed in multiple peer reviewed journal articles. The “world wide web” also has similar disclosures. For example, in a 2019 article, Tridedi, et al. disclose a set of volatile biomarkers specific to Parkinson's Disease.¹ Skin swabs were collected from the upper back areas of Parkinson's Disease patients and a group of controls. The study highlighted detected levels of artemisinic acid, dodecane, eicosane, hexyl acetate, hippuric acid, octacosane, octadecanal, octanal, and perillic aldehyde using thermal desorption-gas chromatography-mass spectrometry (TD-GC-MS) analysis. ¹Discovery of Volatile Biomarkers of Parkinson's Disease from Sebum. Drupad K. Trivedi, Eleanor Sinclair, Yun Xu, Depanjan Sarkar, Caitlin Walton-Doyle, Camilla Liscio, Phine Banks, Joy Milne, Monty Silverdale, Tilo Kunath, Royston Goodacre, and Perdita Barran. ACS Cent. Sci. 2019, 5, 4, 599-606. Mar. 20, 2019. https://doi.org/10.1021/acscentsci.8b00879.

A 2017 paper reported results relating to diagnosis and classification of a plurality of diseases following analysis of exhaled breath.²The 17 diseases with reported results were: lung cancer, colorectal cancer, head and neck cancer, ovarian cancer, bladder cancer, prostate cancer, kidney cancer, gastric cancer, Crohn's disease, ulcerative colitis, irritable bowel syndrome, idiopathic Parkinson's, atypical Parkinsonism, multiple sclerosis, pulmonary arterial hypertension, pre-eclampsia, and chronic kidney disease. 2-ethylhexanol, 3-methylhexane, 5-ethyl-3-methyloctane, acetone, ethanol, ethyl acetate, ethylbenzene, isononane, isoprene, nonanal, styrene, toluene, and undecane were identified as being present in amounts that significantly differed from control groups and/or the other diseases. An “artificially intelligent nanoarray that is based on chemiresistive layers of molecularly modified gold nanoparticles and random network of single-wall carbon nanotubes” and GC-MS were used in analysis and characterization. ²Diagnosis and Classification of 17 Diseases from 1404 Subjects via Pattern Analysis of Exhaled Molecules. Morad K. Nakhleht, Haitham Amal, Raneen Jeries, Yoav Y. Broza, Manal Aboud, Alaa Gharra, Hodaya Ivgi, Salam Khatib, Shifaa Badarneh, Lior Har-Shai, Lea Glass-Marmor, Izabella Lejbkowicz, Ariel Miller, Samih Badarny, Raz Winer, John Finberg, Sylvia Cohen-Kaminsky, Frédéric Perros, David Montani, Barbara Girerd, Gilles Garcia, Gerald Simonneau, Farid Nakhoul, Shira Baram, Raed Salim, Marwan Hakim, Maayan Gruber, Ohad Ronen, Tal Marshak, liana Doweck, Ofer Nativ, Zaher Bahouth, Da-you Shi, Wei Zhang, Qing-ling Hua, Yue-yin Pan, Li Tao, Hu Liu, Amir Karban, Eduard Koifman, Tova Rainis, Roberts Skapars, Armands Sivins, Guntis Ancans, Inta Liepniece-Karele, Ilze Kikuste, leva Lasina, Ivars Tolmanis, Douglas JohnsonOrcid, Stuart Z. Millstone, Jennifer Fulton, John W. Wells, Larry H. Wilf, Marc Humbert, Marcis Leja, Nir Peled, and Hossam Haick Orcid. ACS Nano 2017, 11, 1, 112-125. Dec. 21, 2016. https://doi.org/10.1021/acsnano.6b04930.

Another disease, Alzheimer's Disease (AD) tested the hypothesis “that dysregulation in energy use, mitochondrial abnormalities, oxidative stress, and neuroinflammation that occur with aging are contributing factors to the pathophysiology of AD.”³ In a rat model, an array including 3 VOC sensor elements butylated hydroxytoluene [BHT], pivalic acid and 2,3-dimethylheptane identified rats with an AD modeled mutation. ³Detection of presymptomatic Alzheimer's disease through breath biomarkers. Shadi Emam, Mehdi Nasrollahpour, Bradley Colarusso, Xuezhu Cai, Simone Grant, Praveen Kulkarni, Adam Ekenseair, Codi4 Gharagouzloo, Craig F. Ferris, Nian-Xiang Sun. Alzheimer's & Dementia: Diagnosis, Assessment & Disease Monitoring. 14 Oct. 2020. https://doi.org/10.1002/dad2.12088

Thus, the concept that VOC analysis can be reliable in the diagnoses of diseases, generally, and specifically for neurological disease and autoimmune disease, is accepted in the art.

The present invention builds on these findings and features a device that improves ease and reliability in sample analysis. The device uses state of the art nanosensing elements to analyze the VOCs corralled within the protective environment on the otic canal. The otic canal includes small volume gaseous emissions sourced from circulation including metabolites originating from brain tissue. Assaying these metabolites can be instrumental for rapid assessment of neurologic disease. The present invention provides a non-invasive means for rapidly, even continuously, monitoring metabolites especially those emanating from the brain.

The devices of the invention are analogous to a canine nose with respect to the dog's ability to differentiate odors (vapors) in an ambient gas. The device may therefore be characterized as a “nose” or “sniffer”. While a dog's nose takes human smelling capabilities up several notches, the sniffer device of the present invention is even more sensitive. The nanosensing elements in the sniffer device of the present invention can produce a signal when a single molecule is in close proximity to the sensing surface. A collection of molecules in close approximation to a sensing element surface produces a response indicative of the volatile organic compounds (VOCs) present in the ambient gas. When the source of the ambient gas is tightly controlled, the analysis of VOCs from the controlled sample is specific to the source. Using multiple sensing elements engineered with different specificities or selectivities for interacting molecules provides a multi-dimensional analysis that when analyzed can recognize patterns of VOCs specific to disease or source location.

Specificity/selectivity is modulated by treating or decorating the electronically active surface of each sensor element with a “dopant” or functionalizing compound. The dopants or decorators act to attract and/or repel individual molecules in the immediately present ambient gas. When a volatile organic molecule is proximate to or near a sensing surface the molecule is attracted or repelled by interactions with the sensor surface. The electron clouds of both the molecule and the sensing element respond to one another as their surface electrons repel each other and are attracted to the more positive portions of each. The attraction/repulsion of sensor surface electrons in each sensing element provides the sensor with a change specifically responsive to the close interaction(s). The nanosensing elements disposed on gas accessible surfaces are capable of signaling movement of a single molecule from one element passing over. The provision of differently decorated sensing elements and the multi-dimensional data thereby produced allows pattern recognition relating to several molecular interactions with the differentiated sensing elements to identify patterns of metabolic events and associate such patterns with specific metabolic patterns, e.g., disease states. Monitoring off-gases from an individual is specific to metabolic events that produce those gases. Monitoring gases from a specific location on the body accentuates the analysis.

Surface characteristics of a sensing element will differ depending on amount and structure of the dopant. The surface interactions also depend on a basal charge on the sensor or an underlying surface. Magnetic and electric fields and temperatures may also be modulated to affect interactions and thus if desired may contribute to a signature or profile developed to describe or characterize interactions between molecules in a sample and the sensors.

A gas may be allowed to randomly interact with sensor surfaces, e.g., through a molecule's kinetic energy or temperature component. A convective gas driver may be incorporated into the device to intensify interactions between the otic canal gases and the sensing elements. For example, a heater or heating element may be employed to accelerate gas molecule movement and through expansion of gases cause a pressure gradient that the molecules will migrate across to equilibrate the pressures. Gas may also be moved to contact across the sensing surfaces by causing a bulk flow, e.g., by physically decreasing or increasing pressure in a zone. For example, heating the tip of a probe in the otic canal will increase kinetic energy and pressure from the heated molecules to produce a bulk or convective flow which may be used to facilitate interactions between volatilized molecules and the sensors. A bulk flow may involve minimal net displacement or otic air flushing when a pulse flow is instigated. The pulsing moves small, e.g., microliter volumes of air onto and off the sensor surfaces. A deformable membrane may vibrate to create a pulse of positive and negative pressures locally withing the canal. The pulsatile disturbance results in a greater volume of otic gases contacting the sensor surface than would occur from diffusion or constant or continuous flow.

A stream of gas may be forced into the medial portions of the canal to displace gas already there and to drive it across sensor surfaces. A vacuum (decreased air pressure) may be developed towards or outside the lateral portions or opening of the canal (otic meatus) to create a flow from the canal interior across sensing elements. An outward flow through the central portion of a device may cause an inward flow along the walls of the canal and collect additional volatile compounds. Any acceptable source may drive such flow, e.g., a fan, a volume displacer, a syringe, a transverse flow, etc.

A primary site for sampling is the otic canal. The otic canal, being a semi-enclosed environment, in close communication with the head and brain tissue is a preferred source for monitoring activities in these areas. Gases in the otic canal are not as strongly influenced by ambient gases, food, drink, etc., as, for example, breath or gut gases. The semi-enclosed ear canal lessens mixing with ambient air and therefore is a superior source for reliable, less contaminated sampling.

The volume of gas within the canal turns over slowly and as gases are released within the canal a slow net outflow of gas results. This slow turnover and outward flow allows concentrations of volatile off-gassing from the walls of the canal and the eardrum to achieve a semi-equilibrium state that is a collection of off-gasses emitted over time from the body and less contaminated than gasses that might be sampled off another body surface, such as forearm, armpit, torso, etc. The otic canal is also a source or earwax, a protective secretion lining the canal. Earwax is a source of multiple volatile organic compounds (VOCs) that may be assessed to evaluate metabolism within the body and especially in the head and brain area. The otic coating emits volatile compounds into the gaseous environment within the ear canal from which these compounds can be captured and assayed. Samples may be obtained bilaterally when desired to potentially differentiate severity of disease relating to the left and right sides or brain hemispheres.

VOC detection devices have been described in detail, for example, in U.S. patent application 63/017,693 filed Apr. 30, 2020; the disclosures of which are hereby included in their entireties by reference. Developers are continuously improving the capabilities of electronic noses using tried and true sensors such as metal oxides. See, for example, “Robust and Rapid Detection of Mixed Volatile Organic Compounds in Flow Through Air by a Low Cost Electronic Nose”, by Huang and Wu, published Aug. 21, 2020 wherein acetone, ethanol and isopropyl alcohol were detection targets, indicating that cross referencing a plurality of sensors within an analytical algorithm appears to offer detection advantages.

A preferred sensing device is an extremely compact, high sensitivity device. One such device useful for this invention features single walled carbon nanotubules (SWNTs) exposed on a surface brought in contact and allowed to interact with VOC compounds being evaluated. Other embodiments may feature graphene or synthetic polymers to similar effect. SWNTs and other carbon substrates, such as thin or single layer graphene, provide both a large surface to volume ratio (to facilitate sensor—molecule interaction) and electrical conductivity that facilitate signal transduction. In the April 30 patent application referenced above, nano-sensor elements {NSEs}, each including at least one sensing surface, are capable of, for example, field-effect transistor (FET) or other physico-electrical property/activity. Such structures include, but are not limited to: semi-conducting nano-wires, carbon nano-tubes—including single-wall carbon nano-tubes, chitosan-cantilever based, synthetic polymers—including dendrimers, plasmon resonance nano-sensors, Förster resonance energy transfer nano-sensors, paramagnetic compounds, surface active crystals, vibrational phonon nano-sensors, magnetically resonant compositions, optical emitting or transforming compositions, optical frequency (or wavelength) based nano-sensors (sensitive to photon transmittance, absorption, reflection, energy modulation, etc.).

One preferred format of the present invention may feature “chips” with modular nanosensing elements (or nano-sensor element (NSE) that are independently maintained at a fixed, fluctuating, stochastic, alternating, discontinuous or flashing feeder power supply. The outputs of each NSE may be individually wired to a dedicated data transducer or a selection of sensor outputs may use a common carrier circuit and thus be “averaged”. In some embodiments, a simpler circuitry may involve multiple elements feeding a single output that may sum the outputs to deliver an average reading. When one or more of the “averaged” sensors is turned off or powered down, the average will not include output from these one or more powered down sensors. When input sensors are powered individually, for example, in a cycling pattern when only one (or a selected portion) of the input electrodes being charged, averaged outputs synchronized with the timing of input charging can thus provide data from individual channels.

The single output may connect and thereby collect data signal from any desired fraction of elements. For example, a single output may receive signal from all elements on a chip, half the elements on a chip, one-third the elements on a chip, a quarter the elements on a chip, a fifth the elements on a chip, and so on, for example, ⅙, 1/7, ⅛, 1/9, 1/10, 1/12, 1/20, 1/25, 1/33, 1/50, 1/100, etc. Any output may be associated with 1, 2, 3, 4, 5, 6, 7, 8, 9, 10, 11, 12, 13, 14, 15, 16, 17, 18, 19, 20, 21, 22, . . . , 24, . . . , 32, . . . , 48, . . . , 50, . . . , 64, . . . , 96, . . . , 100, . . . , 128, . . . , 200, . . . , 250, . . . , 256, . . . , 500, . . . , 512, . . . , 1000, . . . , 1024, . . . , 2048, . . . , 4096, . . . , 5000, . . . , 8192, . . . , 10,000 (10⁴), . . . , 16,384, . . . , 2¹⁵, . . . , 2¹⁶, . . . , 10⁵, . . . , 2¹⁷, . . . , 2¹⁸, . . . , 2¹⁹, 10⁶, . . . , 2²⁰, . . . , total number of sensors on a chip which may vary with time or programmed instructions. The precise count of sensor elements associated with any output in general is a design feature and does not define operative functions of the invention. The counts specifically exemplified above are exemplary low numbers of sensors that may feed an output and higher numbers common in conventional plate assays and powers of 2 and 10 frequently used or approximated in biological or chemical science or physics or electronics. Outputs may chronologically rotate periodically outputting data from a fraction of the elements on a chip or with multiple chips, rotating amongst chips.

When connected to multiple elements, the output may average output signals from each, and modulate weightings of elements in an average or in contribution to signature formation. With fluctuating or non-constant inputs, weighting is also controllable. For example, in an extreme sense a stochastic or alternating input, when alternated to off, that element's output will report a zero weighting, or a fluctuating or stochastic feed can serve to physically, rather than mathematically control the weighting output. The designer and/or operator will have options for mathematical/algorithmic or physical/electrical weighting of each NSE input to the data analysis. A group of elements may therefore receive the same feeder voltage, or the feeders may be independently controlled.

Instruction to or control of the system may be through information encoded on a sample package, information encoded on a sensor chip, from a user interface, information provided remotely by machine or active user, or information encoded within the device. For example, samples may be encoded with a shape or mass signal. A sample having a given shape would instruct the device to proceed with the assay that the software associates with that shape. In addition to shape, sample cartridge mass may be instructive as to the sample mass itself or may, perhaps distinguishing a smaller or a larger sample, instruct processing of the sample to allow access at controlled volume or feed rate of the VOCs into analysis. An optically readable signal, (color, transparency, bar code, text, etc.) an electronically accessible signal (RFID, memory chip or drive, etc.), a magnetic signal, etc., are also usable in controlling the device.

Specific control can be through a large variety of means and is not generally to be considered as limiting the invention. The signal embedded, itself, may be adequate to program the relevant machine cycles or may instruct the machine to access further instructions for example, in machine archives or at a remote location. A device may cycle through one or a plurality of signals as directed or required. Chips may be interchangeable and be encoded using signals analogous to those discussed above relating to sample cartridges.

Such device is preferably compact. In a preferred embodiment, an otic gas sample is analyzed in a probe fixture, e.g., a device similar in shape to an otoscope, that collects and analyzes an otic sample, e.g., an earwax offgas sample. Data may be stored and analyzed within the device and/or transmitted to an accessory device for data retention and analysis. In another preferred embodiment, a gas sample or a plurality of samples may be collected for delivery into a remote device by direct streaming from the canal probe to an assay analysis component or may be captured in a pod with physical delivery of the pod to an analytical device.

An otoscope shaped or configured device comprises a funnel shape probe that can be inserted into the ear canal. When actually a scope, it generally includes a light and a magnifier that permits the canal walls and eardrum to be visually inspected. Some scopes use a video camera to view the earparts. The scope is manipulatable using a handle, generally rod shaped, at an angle about or slightly greater than 90°. A collection device may heat the canal to increase VOC release. The heating may be convective, e.g., through flowing air; radiant, e.g., through infrared EM radiation, preferably greater than about 3 microns, more preferably greater than about 7 microns; contact, e.g., a porous bulb that may be warmed using any warming method, preferably with a low specific heat (less than water, perhaps about one quarter, one third, one-half that of water, e.g., around 0.2, 0.3, 0.4, 0.5, 0.6±0.05 (kcal/(kg ° C.)). The heater may be external to the ear canal, but configured to increase the wall temperature. Preferably the heater heats a portion of the device that enters the ear canal and heats a portion medial to the otic meatus.

Off gassing within the canal may be promoted by thermally exciting the VOCs in the inner ear wall to encourage molecular movement and accelerate delivery to and contact with one or more sensing elements. Any manner of controlled heating is acceptable but must be within tissue tolerances. Thermal excitation may similarly be applied to promote deliver of VOCs to and interaction with a sensing element, block, chip, etc., when the gases are analyzed remotely.

The sensing component, which may comprise a chip or a plurality of chips, monitors electrical changes in response to proximity of a VOC to a sensor element of the component. Numerous sensor elements are used to differentiate between VOC molecules interacting with the plurality of elements. A pattern of sensor element-VOC interactions is collected and analyzed to form a signature. Sensing elements may be maintained at a static temperature or may be heated or cooled during an analysis session. Instantaneous temperature of the sensing component and/or ambient vapor may be a factor in or a part of the formed signature. Individual elements may be heated individually. Zones or volumes within the sensing volume may be excited, e.g., by light, ion beam, etc., to enable additional factors to be included in a signature.

The signatures associated with different diseases may include common elements, including one or a group of elements maintaining similar ratios. For example, an autoimmune disease may provoke development of one or more VOCs rooted in the immune process. A signature may be identified as associated with autoimmunity even if the auto-target is not identified. Immune suppression may relieve symptoms by reducing immune attack even in the absence of specific identification or characterization of the disease. A signature relevant to a disease may include extractable features associated with a disease class. The disease signature may share components with several disease or classes of diseases. Disease distinguishing features may be a single or plurality of characterizing VOC pasterns. A ratio between two or more VOCs, rather than simple presence or absence detected in the sample, may serve to indicate presence of disease or to differentiate between diseases.

In autoimmune disease, a receptor targeted by the disease may increase and/or decrease certain activities within a cell under control of the receptor. In the nervous system, there may be a cascading effect wherein a decrease or increase of a neurotransmitter may affect activities of downstream cells or upstream cells involved in a feedback loop. Metabolites of these cells may be a part of a signature associated with the autoimmune disease targeting that receptor or others in the neuropathway(s).

When a cell protein, not necessarily an active receptor, comes under attack, mechanics of the cell plasma membrane may be affected. For example, if a white cell membrane were to be made less deformable, flow of that cell through arterioles or capillaries may aggravate endothelial cells whose VOCs may become part of that disease signature.

The outcome of early methodology of the present invention is a library of signatures. Signatures may be associated with a class (e.g., autoimmune) of diseases, a cell type associated with the disease, a tissue or organ associated with a disease, a recognized disease (e.g., Lupus, Alzheimer's, Parkinson's, Multiple Sclerosis, H₁N₁ flu, gall bladder, etc.), a stage of disease (e.g., pre-symptomatic, multi-location, etc.), cells expressing a specific receptors etc. A patient's signature is compared to library signatures to suggest diagnosis and/or treatment specific for that patient.

In some embodiments, rather than a sampling element being inserted in the canal, a headphone like device may be placed over the external ear(s). This device optionally incorporates a driver device to move the gases. The driver device may slowly exhaust gas helping to drive gas across sensor surfaces. The driver device may comprise a deformable membrane, e.g., a speaker to deliver tones, music, instructions, and/or other sound to the subject. The device bearing a deformable membrane may appear in many formats, for example, as cups similar to those in sound delivery system or sound deadening headphones. A cap with cups situated closer to the ear canal may provide better comfort for longer wear, e.g., during sleep.

Components that cover and/or provide access to canal gases may sport heating capacities that may improve comfort and may be employed to accelerate off-gassing of VOCs. A warming heater may be present in padding material that contacts the head or the confined gasses may be heated, e.g., by a pulse generating device. The headphones may be coupled with a sound content provider, e.g., music, book, news, verbal instructions, blog, etc., selected by the wearer or technician. Sound content may be included in the device or provided from a remote source, preferably wirelessly. A device worn over the ear may or may not itself comprise data analysis and/or data processing capacities. For example, the covering may contain a cartridge, preferably an exchangeable or removable cartridge that captures the emitted VOCs. VOCs may be captured in a gaseous form in a sealed container. The VOCs may also be captured on a solid or gel support that can be removed and delivered to the assay portion of the device. Many fibers are known to capture odors or VOCs. The format is a designers choice and may include natural or artificial membranes or fibers that have been degassed. Materials including, but not limited to: cotton, aliphatic methacrylates, carbon, PVDF, zeolites, microporous silica, aromatic polydivinylbenzenes polystyrenic-polydivinylbenzene matrices, highly cross-linked styrenic polymers, etc. may be formed as capture filters. VOCs may be desorbed or harvested from such filters or capture elements, e.g., using heat, gas or liquid purge or flush, vacuum, CO₂, nitrogen displacement, etc., for presentation to sensor element arrays.

Such deformable membrane may participate in gas delivery to sensors of the device. A bud like device component or headphone like covering may be worn for relatively short assays, e.g., over the order of minutes to longer sample sessions, e.g., 30-45 minutes, 1 or more hours, a half day (about 2, 3 or 4 hours before or after lunch), a full day, e.g., 6, 7, 8 or more hours, including overnight, and occasionally for longer period such as extended EEG sessions or clinic stays.

In another example, a device component fitting around or over the head, may be worn for a predetermined length of time. While the device in some examples incorporates the sensors and electronics for assaying VOCs, in one example, the headset, earmuff, etc., shaped device collects sample(s) that is then delivered to a sensing component that is not physically attached to the head worn component. In a first format, the subject or an assistant removes the head component from its packaging. The component may be adjusted for comfort and/or proper alignment over the ear canal(s). A covering is removed or reconfigured to expose a sampling cartridge to the outside air or eargas. The sampling component is fitted over the head. After a predetermined length of time, the component is removed fro the head with its sampling chamber appropriately resealed. The entire headset package component is repackaged for delivery to the lab for VOC analysis. In some embodiments a cartridge is removable. The cartridge(s) is/are removed from the headset holder and packaged for delivery to the lab for analysis. A cartridge may be retained by any suitable means, e.g., by friction, by one or more clips, by a stretch fiber or crevice, by a screw mechanism, etc. The headset may be retained for insertion of additional cartridge(s) when a retest, e.g., monitoring disease progression is performed. A form fitting head covering or cap extending over the head to cover and seal the ear canals may be substituted for the headset shaped component in some embodiments. If preferred, for example, the cap, bud, headset, may incorporate sensing elements to collect data and may also include processing units for analyzing, storing, and/or transmitting data, either in raw formats or with more complete signature analyses and comparisons. A sample unassayed, partially assessed or more rigorously analyzed for signature comparison, may be retained in a sealable cartridge for subsequent analysis. The cartridge may be sealed and removed when the device is freed from the head either by the user or an assistant. The cartridge may include a temperature control feature, e.g., a capacity to heat the area around or in the ear canal and/or cool a sample retention filter or retainer. The cartridge may incorporate electronic components. The sealing format may be any conventionally used in the art, for example, a snap fit cover, a screw on cover, an elastic cover, a sealable pouch, etc.

A similar process, involves a subject accepting delivery or accessing an earbud or earplug configured device (one or two for a single ear or for a bilateral assay) for insertion in one or both ears. The subject then wears the bud or plug for a predetermined or suggested length of time, removes the plug or bud, and inserts the device(s) in a package to be delivered to a lab where the device(s) are fed into an entry port of a VOC assay device for analysis and characterization.

An alternative format of the present invention comprises a gas driver that draws otic canal gases through the collection portion of the device into an accessory device containing one or more sensing blocks. The gas replacing the gas removed by the device may be ambient air or a selected gas or mixture of gases. For example, an inert gas (may or may not be a noble gas) in provided at a temperature or range of temperatures to optimize testing protocols, e.g., for speed, patient comfort, quality of results.

As the gases are drawn through the accessory device the depth of the otic probe may be adjusted. A low-volume transit tube, e.g., short with small inner diameter, allows obtention of results at a quicker pace and decreases temperature effects from gases flowing into the ear canal when these flows are not otherwise controlled. To the extent that colder air may be annoying to the subject, a heating coil or feeding line may reduce the irritative cold sensation. Slightly warming the air may also be advantageous for subliming or evaporating additional VOCs. Humidity may also be a controlled testing parameter. Polar vapors, water or otherwise, may encourage release or VOCs from the otic coating. Volunteer subjects or patients of different sizes, genders, races, and cultures are tested using probes with flowing gas.

The art mentioned above employed different means for assaying volatile compounds. The different means would be expected to have different sensitivities to different VOCs. In this current example, temperature is a variable that can change the signature profile. Accordingly, a VOC pattern associated with a disease, e.g., Alzheimer's Disease obtained, for example through GC-MS, cannot be assumed to be the same pattern when assayed with another sensor format. Thus, signatures should be clearly identified with the process under which they were obtained.

### EXAMPLES

A panel of patients is chosen. Informed consent is obtained. Patients are associated with one or more disease(s). When patients have agreed, both swab samples (earwax) and otic canal gasses are collected. Samples are analyzed and a signature pattern output is obtained. It is not essential to identify any specific VOC, the sensing pattern obtained by passing the sample over the block provides sufficient distinguishing data even when the chemical structure of one or more of the VOCs is unknown. Patients with a particular diagnosis are associated with that disease. Patients without that diagnosis (but possibly, and most likely, with a diagnosis for one or more other disease(s) can serve as control for each disease in the panel of patients.

While otic gas is targeted for analysis, the process of using the device may involve analyzing a gas sample outside the otic canal as a control factor. The sensor device used for obtaining and analyzing otic gas may be activated outside the canal to collect control samples. For example, a sample of gas from the auricular area, the external meatus, etc., may serve to control for ambient gases the subject may be immersed in.

The data are fed into a processor either in the collection device itself or an associated component which applies artificial intelligence or machine learning to identify portions of each patient's signature may be associated with a particular diagnosis. In some circumstances a part of the signature, e.g., a ratio of VOC A to VOC B may be similar between a plurality of diseases. Gross outputs, the interactions with sets of sensors with a collection of VOCs that pass over each set can be processed and analyzed to form signatures. A multi-channel, e.g., 256 sensor chip bearing, e.g., 16 channels of 16 sensing elements (16×16) can from a pattern of responses in 16 dimensions across a pre-selected sampling time. Rates of signal changes caused by the collection of VOCs passing by each channel can form robust signals for analyzing disease presence, severity, status, and changes in repeated analyses from the same source.

A library of signature patterns is thus collected with characteristic signature elements being associated with a disease as signature for that disease. Earwax and otic canal gasses are separately analyzed but may be correlated or cross-referenced. Left- and right-side readings are cross-referenced and correlated when possible. Differences may be indicative of disease differences between hemispheres, circulatory aberrations, previous injury, sleep positions, headset wearing, etc. Data may show that left-right differences can provide information suggesting lesion location, or may suggest preferences for using the right or left ear for testing depending on the subject's behaviors, habits, and activities.

A second group of patients is similarly evaluated to confirm or adjust disease signatures. The system is then used for diagnosing patients. In preferred practice, over a period of years, the data are periodically reevaluated and refined. For example, patients who are diagnosed with a disease months or years after the initial signature development may have their data reevaluated for potential indication of a pre-disease state or early disease detection. Having a signature library available, a patient comes to clinic and as a part of screening has an otoscope like device inserted in the ear canal.

The device, in this example, actually includes an otoscope function incorporating a light and a view-port. The medical provider uses the optics of the otoscope to center the device within the canal. A gas sample is drawn into and analyzed in the otoscope device. The device communicates the patient's otic gas signature electronically to a home device which displays and/or prints out a report. A clinician then counsels the patient with emphases on current and developing disease(s) that are indicated or suggested through the device's comparison of the patient's VOC signature to the signature library for diseases.

As an alternative to an otoscope like device to access the otic canal, a probe shaped similar to an earbud or earplug may be inserted into the ear to access otic gases. The earbud or earplug shaped device may be self contained, e.g., including an integral power source, sensor surfaces, data processor, amplifier, data storage, a signal transducer, and/or a communication interface. Such bud may remain in the ear for an extended period reducing or minimizing a preference for a gas driving component.

In some embodiments, rather than a sampling element being inserted in the canal, a headphone like device may be placed over the external ear(s). This device optionally incorporates a driver device to move the gases. The driver device may slowly exhaust gas helping to drive gas across sensor surfaces. The driver device may comprise a deformable membrane, e.g., a speaker to deliver tones, music, instructions, and/or other sound to the subject. The device bearing a deformable membrane may appear in many formats, for example, as cups similar to those in sound delivery system or sound deadening headphones. A cap with cups to situated closer to the ear canal may provide better comfort for longer wear, e.g., during sleep. Components that cover and/or provide access to canal gases may sport heating capacities that may improve comfort and may be employed to accelerate off-gassing of VOCs. A warming heater may be present in padding material that contacts the head or the confined gasses may be heated, e.g., by a pulse generating device. The headphones may be coupled with a sound content provider, e.g., music, book, news, verbal instructions, blog, etc., selected by the wearer or technician. Sound content may be included in the device or provided from a remote source, preferably wirelessly. A device worn over the ear may or may not itself comprise data analysis and/or data processing capacities. For example, the covering may contain a cartridge, preferably an exchangeable or removable cartridge that captures the emitted VOCs.

VOCs may be captured in a gaseous form in a sealed container. The VOCs may also be captured on a solid or gel support that can be removed and delivered to the assay portion of the device. Many fibers are known to capture odors or VOCs. The format is a designers choice and may include natural or artificial membranes or fibers that have been degassed. Materials including, but not limited to: cotton, aliphatic methacrylates, carbon, PVDF, zeolites, microporous silica, aromatic polydivinylbenzenes polystyrenic-polydivinylbenzene matrices, highly cross-linked styrenic polymers, etc. may be formed as capture filters. VOCs may be desorbed or harvested from such filters or capture elements, e.g., using heat, gas or liquid purge or flush, vacuum, CO₂, nitrogen displacement, etc., for presentation to sensor element arrays.

Such deformable membrane may participate in gas delivery to sensors of the device. A bud like device component or headphone like covering may be worn for relatively short assays, e.g., over the order of minutes to longer sample sessions, e.g., 30-45 minutes, 1 or more hours, a half day (about 2, 3 or 4 hours before or after lunch), a full day, e.g., 6, 7, 8 or more hours, including overnight, and occasionally for longer period such as extended EEG sessions or clinic stays.

In another example, a device component fitting around or over the head, may be worn for a predetermined length of time. While the device in some examples incorporates the sensors and electronics for assaying VOCs, in this example, the headset shaped device collects sample(s) that is then delivered to a sensing component that is not physically attached to the head worn component. In a first format, the subject or an assistant removes the head component from its packaging. The component may be adjusted for comfort and/or proper alignment over the ear canal(s). A covering is removed or reconfigured to expose a sampling cartridge to the outside air or eargas. The sampling component is fitted over the head. After a predetermined length of time, the component is removed fro the head with its sampling chamber appropriately resealed. The entire headset package component is repackaged for delivery to the lab for VOC analysis. In some embodiments a sample collector module provides a port for a cartridge that is removable or exchangeable. The cartridge(s) is/are removed from the headset holder and packaged for delivery to the lab for analysis. A cartridge may be retained by any suitable means, e.g., by friction, by one or more clips, by a stretch fiber or crevice, by a screw mechanism, etc. The headset may be retained for insertion of additional cartridge(s) when a retest, e.g., monitoring disease progression is performed. A head covering or cap may be substituted for the headset shaped component in some embodiments.

A similar process, involves a subject accepting delivery or accessing an earbud or earplug configured device (one or two for a single ear or for a bilateral assay) for insertion in one or both ears. The subject then wears the bud or plug for a predetermined or suggested length of time, removes the plug or bud, and inserts the device(s) in a package to be delivered to a lab where the device(s) are fed into an entry port of a VOC assay device for analysis and characterization.

An alternative format of the present invention comprises a gas driver that draws otic canal gases through the collection portion of the device into an accessory device containing one or more sensing blocks. The gas replacing the gas removed by the device may be ambient air or a selected gas or mixture of gases. For example, an inert gas (may or may not be a noble gas) in provided at a temperature or range of temperatures to optimize testing protocols, e.g., for speed, patient comfort, quality of results.

As the gases are drawn through the accessory device the depth of the otic probe may be adjusted. A low-volume transit tube, e.g., short with small inner diameter, allows obtention of results at a quicker pace and decreases temperature effects from gases flowing into the ear canal when these flows are not otherwise controlled. To the extent that colder air may be annoying to the subject, a heating coil or feeding line may reduce the irritative cold sensation. Slightly warming the air may also be advantageous for subliming or evaporating additional VOCs. Humidity may also be a controlled testing parameter. Polar vapors, water or otherwise, may encourage release or VOCs from the otic coating. Volunteer subjects or patients of different sizes, genders, races, and cultures are tested using probes with flowing gas.

The art mentioned above employed different means for assaying volatile compounds. The different means would be expected to have different sensitivities to different VOCs. In this current example, temperature is a variable that can change the signature profile. Accordingly, a VOC pattern associated with a disease, e.g., Alzheimer's Disease obtained, for example through GC-MS, cannot be assumed to be the same pattern when assayed with another sensor format. Thus, signatures should be clearly identified with the process under which they were obtained.

Nano FETs and other nano-sensor formats generally operate by changing electrical properties as a substance comes in close proximity to the sensor. The interaction between electrons of the sensed molecule and the sensor surface perturbs the steady state of that surface to elicit its signal. The altered distribution of electrons induced by a proximal molecules, (depending on the design of the nano-sensor) changes one or more electrical properties, e.g., impedance, resistance-conductivity, capacitance, inductance, etc. and thus the physical movement of a detectable particle, e.g., an electron, a photon, etc.

Specificity of coordination (interaction) between sensor surface and VOC molecule may be provided by functionalizing or decorating the carbon gate electrode. For example, many sequences of nucleic acid such as DNA or RNA will stringently coordinate or bind with the SWNT structure. These nucleic acids may be naturally occurring or synthetic. The ringed structures of the nucleic acids or other molecules such as peptides containing a large fraction of ringed structures associate strongly with the nanotubular structures. These functionalizing, or decorating, additions to the SWNTs serve to selectively capture proximal molecules. When the chemical geometry is changed, the gating characteristic of the associated carbon bridging the input and output electrodes is modulated. Differently decorated or heated elements respond differently different proximal VOC. A single element may be associated with a single sequence or a plurality of functionalizing sequences. Output characteristics of gating in response to one or more gaseous compounds, e.g., VOCs are then collated into a data library. When that NSE responds in the same manner, presence of the VOC is confirmed. Stringent selection of element functionalizations, and subsequent application of the controllable assay variables can optimize certainty of VOC identification at a desired level, for example, increasing manipulation of the variable parameters can achieve certainty of 99+%. In special circumstances, for example to develop rapid profiling of a new VOC signature (i.e., pathogen), a simplified screening protocol or developmental process may begin with a lower level of certainty, e.g., 85%, 95%, etc. Subsequent refinements then could be applied to raise the level of certainty until reaching a mathematical and chemical sensitivity to an acceptable level, e.g., a 99+% certainty. while also minimizing false positives

A single element may be capable of indicating the presence of more than one compound. For example, similar compounds may not be distinguished in their association/coordination with the element surface and therefore may in certain circumstances produce indistinguishable signals on their own. But the single element may, for example, in conjunction with one or more other elements provide definitive results with respect to the VOCs that may interact with any one element. Alternatively, the single element when operated at a different temperature, voltage or other variable may distinguish between the different compounds binding the element under static conditions. The discussion above describing the variable inputs and input patterns and different resulting outputs relates to such differentiation capabilities.

In another example, an individual is identified as a candidate for assessment of VOCs from the head area The assessment is to be derived from inner ear gases. A cap similar in shape to the protective headgear worn in water polo matches is provided. The version in this example is a Lycra or elastic fabric based form-fitting headgear. Where the water polo cap incorporates rigid or semi-rigid cups that protect the ears from impacts or shear forces, the device of the present invention incorporates the VOC component. The VOC component can take on different formats depending on the choice of the user(s). The cup portion may be rigid, semi-rigid, padded or semi-soft. It may be integral in the headgear. Preferably the cup is identical in design save in some embodiments a marking to designate left or right. In some embodiments the attachment means will only allow a side designated component to be attached to the designated side. Attachment in a primary example uses a simple slot for alignment with a turn to secure the device. A tab may be incorporated to prevent accidental release. A screw-in or snap-in format may be provided as alternative selections.

The cup may contain a solid or gel to adsorb VOCs from the cup (ear) area. More elegant examples include one or more features selected from the group consisting of: a sound speaker that may provide entertainment, a lesson, and/or instruction; a slow pulsing gas driver to reduce and increase air pressure movement of VOCs to the retainer; a heater to stabilize temperature and/or to increase VOC emission; VOC sensors; a data collector; a data processor; a data receiver and/or transmitter; an alarm (e.g., to signal end of trial); etc.

The subject wears the device for a preselected period of time, for example the length of an office or clinic visit, the length of a movie, a sleep period (e.g., overnight), etc.

Embodiments may incorporate accessories including, but not limited to: a convective gas driver for introducing gas into the otic canal; a convective gas driver driving gas from the otic canal; a gas driver to move the air that is selected from the group consisting of: a syringe, a fan, and a pump; etc.

Embodiments may incorporate a sample collector module for sample retention for historical or remote analysis. Some embodiments may be shaped as or incorporated in a headgear that covers at least one otic meatus.

The device may feature one or more pulsatile gas drivers for driving gas in said otic canal in a pulsatile fashion. The pulsation may result from a deformable membrane, a cycling valve, etc. A sample cartridge holder may help contain or support a collector/retention cartridge which may be associate with a cartridge sealer to isolate the sample(s). The unit or parts thereof can be similar in configuration to an otoscope, an earbud, an earplug, etc., optionally featuring a heater to heat a portion internal to the otic meatus. Such heater may be associated with a pulsatile air driver for convective air heating, may be radiant heat, and/or may contact the skin.

Since the manner through which a signature is obtained is determinative of the signature outcome, a different assay technique or device format thus requires validation processes to form and confirm VOC signatures for each disease of interest. While animal models may be illustrative, human data are preferred for better relevance to human diseases.

1. A sniffer device for early stage autoimmune and/or neurodegenerative disease detection, said device comprising:
a portal, sized to enter the ear canal and configured to collect gas therefrom;
a passage connecting said portal to an array of nanosensing elements sensitive to at least one volatile organic compound (VOC) flowing through said passage to become available to said array;
said array configured to produce data output in response to interaction with said at least one VOC;
a processor receiving said data output, said processor processing said data output to form a profile or signature characterizing the VOC content within said ear canal; and
an interface capable of outputting said signature or profile for further analysis.

2. The sniffer device of claim 1, further comprising a data analyzer capable of receiving a signature or profile developed by the device.

3. The sniffer device of claim 2 further comprising an interface with a library of profiles or signatures associated with at least one disease.

4. The sniffer device of claim 3 wherein said library comprises profiles or signatures of a plurality of diseases.

5. The sniffer device of claim 1, further comprising a heater component.

6. The sniffer device of claim 5 wherein said heater component operates using radiant heat.

7. The sniffer device of claim 5 wherein said heater component operates using a laser heating element.

8. The sniffer device of claim 5 wherein said heater component operates using resistive heating element.

9. The sniffer device of claim 1 comprising a sensing surface external to the otic meatus.

10. The sniffer device of claim 9 further comprising a tube extending from said portal to said array of nanosensing elements.

11. A sniffer device for early stage autoimmune and neurodegenerative disease detection, said device comprising:
a shield, sized and shaped to enclose or cover the ear canal and configured to collect gas therefrom;
a passage through said shield to an array of nanosensing elements sensitive to at least one volatile organic compound (VOC) flowing through said passage to become available to said array;
said array configured to produce data output in response to interaction with said at least one VOC;
a processor receiving said data output, said processor processing said data output to form a profile or signature characterizing the VOC content within said ear canal; and
an interface capable of outputting said signature or profile for further analysis.

12. The sniffer device of claim 11 further comprising a cartridge comprising a VOC adsorbent.

13. The sniffer device of claim 12 wherein said cartridge is removable.

14. The sniffer device of claim 12 wherein said VOC adsorbent comprises a compound or composition selected from the group consisting of: cotton, aliphatic methacrylates, carbon, PVDF, zeolites, microporous silica, aromatic polydivinylbenzenes polystyrenic-polydivinylbenzene matrices, and highly cross-linked styrenic polymers.

15. The sniffer device of claim 11 further comprising a deformable membrane.

16. The sniffer device of claim 14 wherein said deformable membrane functions as a pulsatile gas driver.

17. The sniffer device of claim 11 said shield comprises a heated enclosing portion.

18. The sniffer device of claim 17 wherein said headgear is in a form selected from the group consisting of: a headset, form fitting cap, and earmuffs.

19. The sniffer device of claim 18 wherein said collector module comprises a port for a removable cartridge.

20. The sniffer device of claim 12 wherein said VOC adsorbent comprises a compound or composition selected from the group consisting of: cotton, aliphatic methacrylates, carbon, PVDF, zeolites, microporous silica, aromatic polydivinylbenzenes polystyrenic-polydivinylbenzene matrices, and highly cross-linked styrenic polymers.
